(19) United States
(12) Patent Application Publication
(10) Pub. No.: US 2005/0251180 A1
(43) Pub. Date: Nov. 10, 2005

(54) Intravascular vessel anastomosis device

(75) Inventors: Lucas Burton, Orleans, IN (US); Amir K. Durrani, Cincinnati, OH (US); Benjamin Hoagland, Birmingham, AL (US); Santosh Tumkur, Cincinnati, OH (US)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(73) Assignee: Vanderbilt University, Nashville, TN (US)

(21) Appl. No.: 11/103,365

(22) Filed: Apr. 11, 2005

Related U.S. Application Data
(60) Provisional application No. 60/561,360, filed on Apr. 12, 2004.

(51) Int. Cl. A61F 2/06

(52) U.S. Cl. 606/155; 623/1.15; 623/1.36

(57) ABSTRACT

An anastomosis device. In one embodiment, the device includes a base member having a first end, a second end, and a body portion with a length defined between the first end and the second end. The body portion is formed with a first edge portion and a second edge portion, where the first edge portion and the second edge portion are apart from each other and defines a recess therebetween and an opening thereof. The device further include a stent member having a substantially tubular body with a longitudinal axis, A, and a lumen defined by the substantially tubular body, and projecting away from the base member such that to form an angle, α, between the base member and the longitudinal axis A of the substantially tubular body, where the lumen of the stent member is in fluid communication with the recess of the base member through the opening.

### CROSS-REFERENCE TO RELATED PATENT APPLICATION

This application claims the benefit, pursuant to 35 U.S.C. §119(e), of provisional U.S. patent application Ser. No. 60/561,360, filed Apr. 12, 2004, entitled “AN INTRAVASCULAR VESSEL ANASTOMOSIS DEVICE,” by Amir Durrani, Lucas Burton, Santosh Tumkur and Ben Hoagland, which is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [5] represents the 5th reference cited in the reference list, namely, Arom K., et al. Safety and efficacy of off-pump coronary artery bypass grafting. Annals of Thoracic Surgery, 2000; 69:704-710.

### FIELD OF THE INVENTION

The present invention generally relates to a medical device and in particular to an anastomosis device for connecting a graft vessel to a target vessel.

### BACKGROUND OF THE INVENTION

Seven million Americans suffer from coronary heart disease, the most common form of heart disease. Coronary artery disease is the single leading cause of death in the United States today [1, 2]. More than 95% of all coronary artery disease is due to arteriosclerosis, clogging of the coronary arteries by cholesterol or calcium deposits [3]. The coronary arteries deliver a constant flow of blood to the heart muscle providing it with a necessary supply of oxygen and nutrients. When these arteries narrow or clog, they cannot provide adequate blood flow to the heart, resulting in coronary heart disease, which includes myocardial infarction (heart attack) and other diseases. One type of treatments for coronary artery disease is coronary artery bypass grafting (hereinafter “CABG”) surgery.

The CABG procedure utilizes a vessel to carry blood around the obstruction in the coronary artery. The vessels used for the CABG are the saphenous vein from the leg or the internal thoracic artery from the inside of the chest. During the procedure, an incision is made down the front of the chest through the sternum exposing the heart and aorta. This incision is called a median sternotomy. The saphenous vein is attached at one end to the aorta and at the other end to the coronary artery downstream of the blockage. When using the internal thoracic artery, the vessel is only severed at one end, and that end is then attached to the coronary artery downstream of the blockage [4].

The bypass surgery includes conventional (arrested heart) surgery and beating heart (off-pump) surgery. In a conventional bypass surgery, a patient is placed on a heart-lung machine, which oxygenates and pumps the blood. The blood is routed outside of the body through the heart-lung machine. During this process, the patients may require transfusions to replenish blood volume, red blood cells, or platelets. The conventional bypass surgery also requires considerable resources in terms of number of staff, products and equipment, medications, and other items that may further impact the total cost of the procedure.

The beating heart bypass surgery is performed on a beating heart through an incision down the middle of the chest without hindering its ability to beat and pump blood. Positioning and stabilization instruments lift and hold the heart and then stabilize a portion of the heart's surface where the bypass graft will be sutured in place, when the heart continues to efficiently beat and pump blood. Some stabilizers use gentle suction to address the primary challenge in beating heart surgery, making it possible to access all surfaces of the heart while reducing motion of the small area of the surface tissue where the surgeon is sewing the bypass graft. Beating heart surgery potentially costs less than conventional surgery at least because cardiopulmonary bypass equipment is not used, and fewer blood products are needed. It is found that the off-pump CABG surgery carries a significantly lower mortality rate in the high-risk population than conventional CABG surgery [5].

However, there are a number of bottlenecks that hinder control CABG efficiency. One bottleneck is associated with insufficient stabilization at an anastomosis site. Another, more pervasive, bottleneck deals with difficulties related to suturing of a harvested vessel to a coronary artery. The degrees of freedom associated with effective stabilization increase dramatically if the suturing process is either made easier for the surgeon or eliminated fully via using an anastomosis device.

On the other hand, vein graft atherosclerosis is a major limitation of arterial bypass surgery. The graft that is used to bypass blood around a blockage in a coronary artery may begin to accumulate blockage at both the proximal and the distal anastomosis sites after the arterial bypass surgery.

Therefore, a heretofore unaddressed need still exists in the art to address the aforementioned deficiencies and inadequacies.

### SUMMARY OF THE INVENTION

The present invention, in one aspect, relates to an anastomosis device for connecting a free open end of a graft vessel to a wall of a target vessel.

In one embodiment, the anastomosis device includes a base member having a first surface, a second surface, a semicircular cylindrical wall formed therebetween the first surface and the second surface, and an opening formed in the semicircular cylindrical wall. The base member further has a first end and a second end defining a base length. Each of the first end and the second end of the base is rounded with a radius. The base member is adapted such that when the base member is introduced into the target vessel, a force directing outwardly from a central axis of the target vessel is perpendicularly applied to the first surface of the base member to bring the second surface of the base member into contact with an inner surface of the wall of the target vessel.

Furthermore, the anastomosis device includes a stent member having an inner surface, an outer surface, a cylindrical wall formed therebetween the inner surface and the outer surface and a lumen formed by the inner surface, and outwardly projecting away from the second surface of the base member along a direction having an angle, α, relative to the base member such that the lumen of the stent member is in fluid communication with the opening in the semicircular cylindrical wall of the base member. In one embodiment, the stent member further comprises a number of rings protruding outwardly from the outer surface of the stent member with each ring at a predetermined position. Each of the number of rings has a semicircular cross-section with a diameter.

Moreover, the anastomosis device includes a layer of chemical composition coated at least on a part of the first surface of the base member and the inner surface of the stent member, respectively. The coated chemical composition is adapted for reducing restenosis and thrombosis simultaneously. In one embodiment, the coated chemical composition includes a cytostatic drug component. The cytostatic drug component in one embodiment includes one of sirolimus, heparin, tacrolimus and any combination thereof.

In operation, the stent member is inserted in the graft vessel through the free open end and the base member is introduced into the target vessel from an opening in the wall of the target vessel so as to anastomose the free open end of the graft vessel to the wall of the target vessel such that the lumen of the graft vessel is in fluid communication with the lumen of the target vessel. The coated chemical composition is gently released to its surrounding environment so as to reduce restenosis in the lumens of the graft vessel and the target vessel, respectively.

In one embodiment the free open end of the graft vessel is secured to the wall of the target vessel at an anastomosis site in which the device is placed with biocompatable glue. In another embodiment, the free open end of the graft vessel is secured to the wall of the target vessel at an anastomosis site in which the device is placed with suture.

The device may be made of a biocompatable material. In one embodiment, the device is formed in one of a solid form, a porous form, a meshwork form, and any combination thereof.

In another aspect, the present invention relates to an anastomosis device. In one embodiment, the anastomosis device includes a base member and a stent member.

The base member has a first surface, a second surface, a base wall formed therebetween the first surface and the second surface, and an opening formed in the base wall. In one embodiment, the base wall of the base member has a semicircular cylindrical form. The base wall of the base member, in another embodiment, has a circular cylindrical form. The base wall of the base member has a thickness defined therebetween the first surface and the second surface of the base member, which can be uniform or non-uniform.

The stent member includes an inner surface, an outer surface, a tubular wall formed therebetween the inner surface and the outer surface and a lumen formed by the inner surface, and is outwardly projecting away from the second surface of the base member along a direction that has an angle, α, relative to the base member such that the lumen of the stent member is in fluid communication with the opening in the base wall of the base member. In one embodiment, the tubular wall of the stent member has a thickness defined therebetween the inner surface and the outer surface of the stent member that is non-uniform along the projecting direction.

The device is formed of a biocompatable material. In one embodiment, the device is at least partially coated with a layer of chemical composition, where the coated chemical composition includes a cytostatic drug component. The cytostatic drug component in one embodiment includes one of sirolimus, heparin, tacrolimus and any combination thereof. The coated chemical composition is adapted for reducing restenosis and thrombosis simultaneously.

In yet another aspect, the present invention relates to an anastomosis device. In one embodiment, the anastomosis device has a base member having a first end, a second end, and a body portion with a length defined between the first end and the second end, where the body portion is formed with a first edge portion and a second edge portion, the first edge portion and the second edge portion being apart from each other defining a recess therebetween, and an opening thereof. In one embodiment, the body portion of the base member with a curvature has a semicircular cross-section.

The anastomosis device also has a stent member having a substantially tubular body with a longitudinal axis, A, and a lumen defined by the substantially tubular body, and projecting away from the base member such that to form an angle, α, between the base member and the longitudinal axis, A, of the substantially tubular body, where the lumen of the stent member is in fluid communication with the recess of the base member through the opening. The angle α is greater than zero but smaller than 180°. The stent member further comprises one or more rings protruding outwardly from the outer surface of the stent member.

The anastomosis device further has a layer of chemical composition coated at least on a part of the base member and the stent member, respectively, where the coated chemical composition is releasable for reducing restenosis and thrombosis simultaneously. The coated chemical composition includes a cytostatic drug component. In one embodiment, the cytostatic drug component includes one of sirolimus, heparin, tacrolimus and any combination thereof.

In a further aspect, the present invention relates to an anastomosis device. In one embodiment, the anastomosis device includes means for performing an anastomosis to connect a wall of a target vessel in fluid communication with a lumen in the target vessel through an opening in the wall of the target vessel, and means for releasing a chemical composition for reducing restenosis during and/or after the anastomosis.

In one embodiment, the means for performing an anastomosis has a base member having a first end, a second end, and a body portion with a length defined between the first end and the second end, where the body portion is formed with a first edge portion and a second edge portion, the first edge portion and the second edge portion being apart from each other defining a recess therebetween, and an opening thereof. In one embodiment, the body portion of the base member with a curvature has a semicircular cross-section. The means for performing an anastomosis further has a stent member having a substantially tubular body with a longitudinal axis, A, and a lumen defined by the substantially tubular body, and projecting away from the base member such that to form an angle, α, between the base member and the longitudinal axis, A, of the substantially tubular body, where the lumen of the stent member is in fluid communication with the recess of the base member through the opening. In one embodiment, the stent member further comprises one or more rings protruding outwardly from the outer surface of the stent member.

In one embodiment, the means for releasing a chemical composition comprises a layer of chemical composition coated at least on a part of the base member and the stent member, respectively, where the coated chemical composition is releasable for reducing restenosis and thrombosis simultaneously. In one embodiment, the coated chemical composition includes a cytostatic drug component. The cytostatic drug component in one embodiment includes one of sirolimus, heparin, tacrolimus and any combination thereof.

In yet a further aspect, the present invention relates to a method for anastomosing a free end of a graft vessel to a wall of a target vessel, wherein the graft vessel has a lumen formed therethrough and the target vessel has a lumen formed therethrough. In one embodiment, the methods includes the step of providing a device having a base member having an opening and a stent member having a lumen formed therethrough and projecting away from the base member such that the lumen of the stent member is in fluid communication with the opening in the base member. The method further includes the steps of inserting the stent member of the device into the graft vessel through the free end of the grafted vessel, whereby the lumen of the stent member is approximately coincident with the lumen of the graft vessel, incising an opening in the wall of the target vessel at an anastomosis site to which the graft vessel is secured, introducing the base member of the device into the target vessel through the opening in the wall of the target vessel such that the lumen of the target vessel is in fluid communication with the lumen of the stent member of the device through the opening in the base wall of the base member of the device, and securing the free end of the graft vessel to the wall of the target vessel at the anastomosis site. In operation, the lumen of the graft vessel is in fluid communication with the lumen of the target vessel through the device.

Additionally, the device is at least partially coated with a layer of chemical composition, and the method includes the step of slowly releasing the coated chemical composition from the device to its surrounding environment. The coated chemical composition is adapted for reducing restenosis and thrombosis simultaneously. In one embodiment, the coated chemical composition includes a cytostatic drug component. The cytostatic drug component in one embodiment includes one of sirolimus, heparin, tacrolimus and any combination thereof.

In one embodiment, the inserting step includes the step of laterally suturing the graft vessel in a circular fashion around the stent member of the device. The securing step, in one embodiment, is performed with biocompatible glue. In another embodiment, the securing step is performed with suture.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 shows an anastomosis device according to one embodiment of the present invention: (A) a perspective view, (B) a front view, and (C) a side view.

FIG. 2 shows schematically a cross-section view of the anastomosis device shown in FIG. 1 in operation: (A) the anastomosis device inserting into a graft vessel, and (B) the anastomosis device connecting the graft vessel to a target vessel.

FIG. 3 shows schematically a procedure of connecting a graft vessel to a target vessel according to one embodiment of the present invention: (A) an anastomosis device having a base member and a stent member, (B) the graft vessel inserted into stent member of the anastomosis device, (C) the target vessel having an opening incised, (D) the graft vessel brought in contact with the target vessel by the anastomosis device, (E) and (F) the graft vessel connected to the target vessel.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise.

The description will be made as to the embodiments of the present invention in conjunction with the accompanying drawings 1-3. In accordance with the purposes of this invention, as embodied and broadly described herein, this invention, in one aspect, relates to an anastomosis device for connecting a graft vessel to a target vessel. In a CABG surgery, the graft vessel is either a saphenous vein from a leg of a living subject or an internal thoracic artery from the inner chest wall of the living subject. The living subject can be a human patient or an animal. When a saphenous vein is used as the graft vessel, the saphenous vein is attached at its one free open end to the aorta and at the other free open end to the coronary artery downstream of the blockage. When the graft vessel is an internal thoracic artery, the internal thoracic artery is only severed at its one free open end, and this end is then attached to the coronary artery downstream of the blockage.

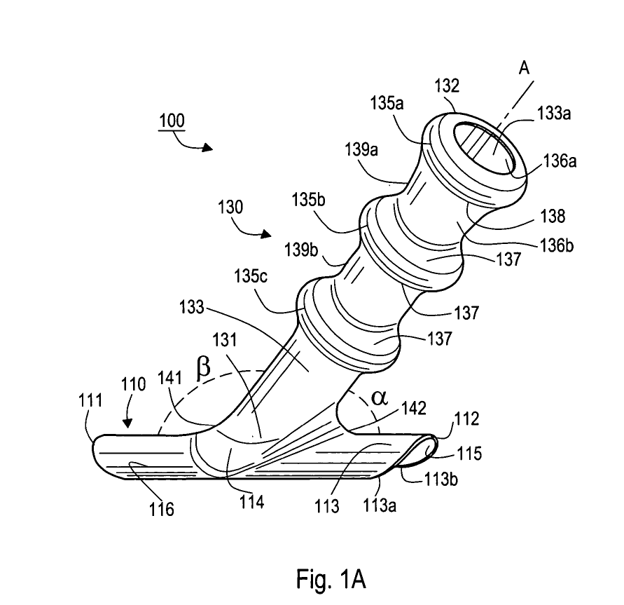

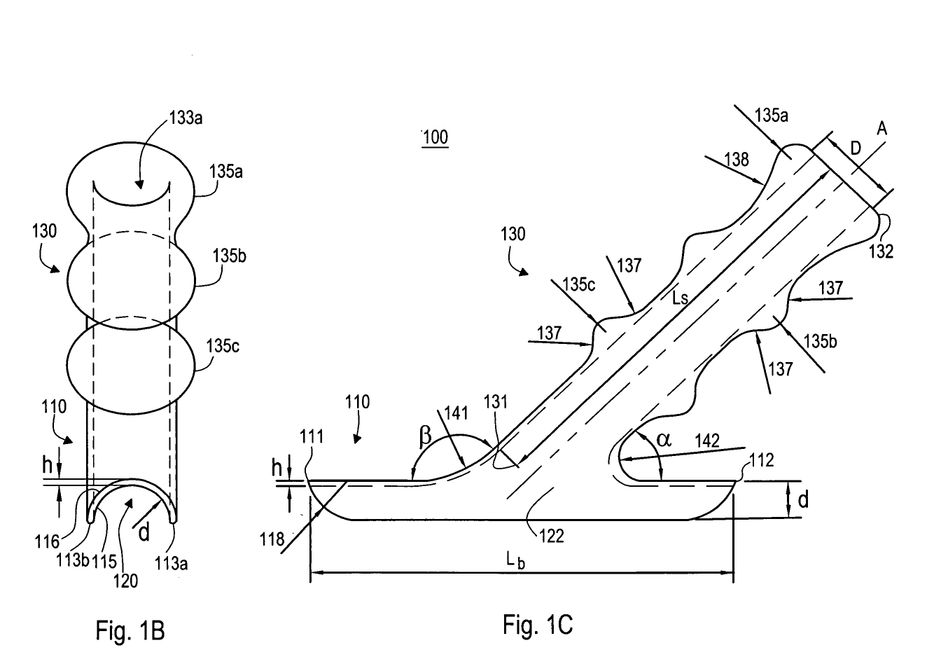

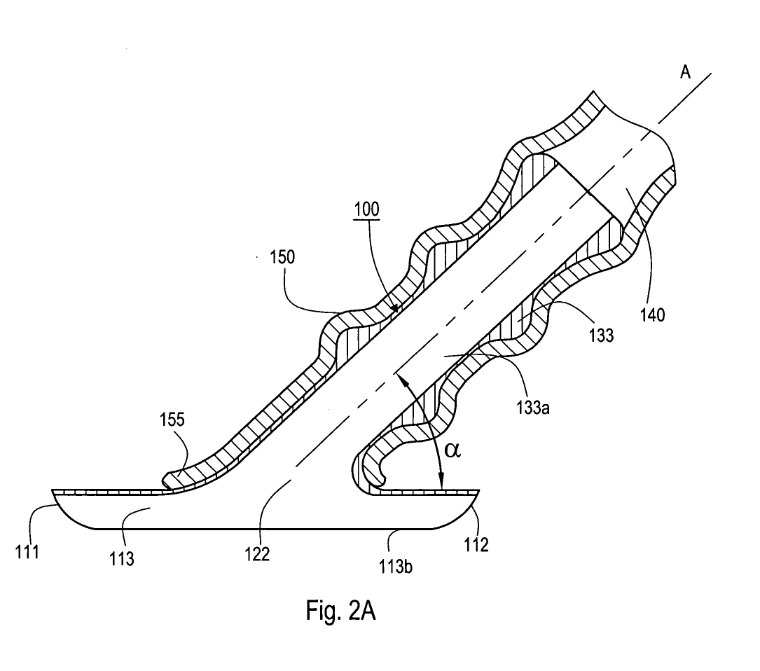

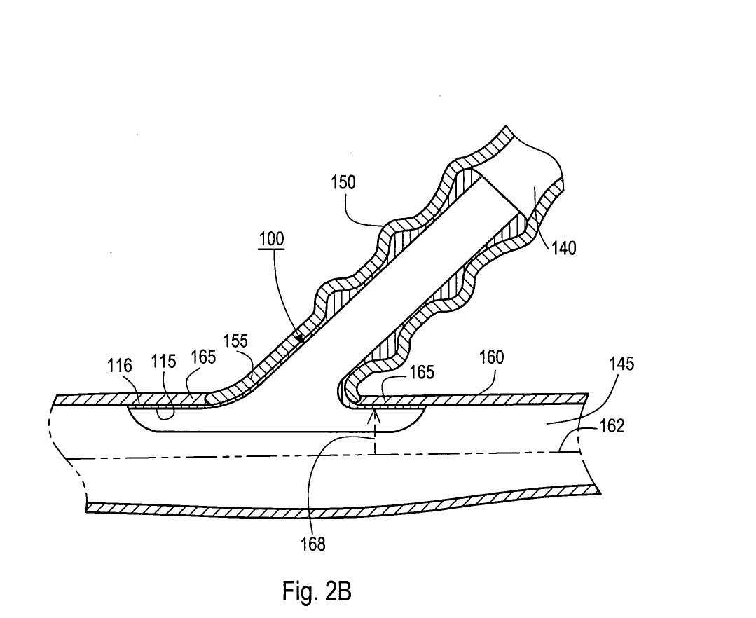

Referring in general to FIGS. 1A-1C, 2A and 2B, and in particular to FIGS. 1A-1C, an anastomosis device 100 includes a base member 110. The base member 110 has a first surface 115, an opposite, second surface 116, a base body portion 113 formed therebetween the first surface 115 and the second surface 116, and an opening 122 formed in the base body portion 113 at a predetermined position. In one embodiment, the opening 122 is formed at the geometrical center 114 of the base body portion 113. The base member 110 further has a first end portion 111 and an opposite, second end portion 112 defining a base length, L_{b}, therebetween. In one embodiment, the base length L_{b} is about 10 mm. Each of the first end portion 111 and the second end portion 112 of the base member 110 is rounded with a radius 118, which is about 1.57 mm in one embodiment. The base member 110 also has a thickness, h, defined therebetween by the first surface 115 and the second surface 116 of the base member 110. The thickness h of the base member 110 is uniform over the base body portion 113 and is about 0.1 mm in one embodiment. The base body portion 113 includes a first edge portion 113a and a second edge portion 113b, which are apart from each other. The base body portion 113, the first edge portion 113a and the second edge portion 113b define a recess 120 therebetween the first edge portion 113a and the second edge portion 113b of the base body portion 113.

In one embodiment, the base body portion 113 is formed in a form of the shell-like semicircular cylindrical wall with a radius, d, substantially around 1 mm, as shown particularly in FIG. 1B. In another embodiment, the base body portion of the base member is formed in a form of hollow cylindrical tube (not shown). The base member 110 can be formed with other dimension parameters.

Furthermore, the anastomosis device 100 includes a stent member 130. The stent member 130 has an inner surface 136a, an outer surface 136b, a cylindrical wall 133 formed therebetween the inner surface 136a and the outer surface 136b and a lumen 133a defined by the inner surface 136a therethrough. The stent member 130 also has an axis, A, which is coincident with an axis of the lumen 133a of the stent member 130. The lumen 133a of the stent member 130 has a diameter, D, which, in one embodiment, is about 2 mm. The stent member 130 outwardly projects away from the second surface 116 of the base member 110 along a direction having an angle, α, relative to the base member 110, where the direction is parallel to the axis A of the stent member 130. The angle α is greater than zero but smaller than 180°. In the embodiment shown in FIG. 1, the angle α=45°, and a junction edge 142 of the stent member 130 and the base member 110 at the angle α=45° is rounded with an inverse radius of about 0.6 mm. A junction edge 141 of the stent member 130 and the base member 110 at an angle β=(180°−α)=135° is rounded with an inverse radius of about 3 mm. The rounded junction profiles between the base member 110 and the stent member 130 allow for a smooth attachment of the graft (harvested) vessel to the coronary artery. The stent member 130 also has a first end 131 merging into the base member 110 and an opposite, second end 132 defining a length, Lₛ. In one embodiment, the length Lₛ of the stent member 130 is about 10.33 mm.

The stent member 130 further includes a plurality of protrusions protruding outwardly from an outer surface 136b of the stent member 130. In one embodiment, the plurality of protrusions is in the form of rings 135a-135c, respectively, as shown in FIGS. 1A-1C. More particularly, ring 135a is located on the second end 132 of the stent member 130, ring 135b is located therebetween the ring 135a and the first end 131 of the stent member 130, and ring 135c is located therebetween the ring 135b and the first end 131 of the stent member 130. Each of the plurality of rings 135a-135c has a semicircular cross-section with a diameter about 1 mm. In one embodiment, junction edges 137 between the ring 135b (135c) and the outer surface 136b of the stent member 130 are rounded at an inverse radius about 1 mm, while junction edge 138 between the ring 135a and the outer surface 136b of the stent member 130 are rounded at an inverse radius about 3 mm. Ring 135a may function as a seal to prevent blood flow from escaping outside the device to a thoracic cavity. An area 139a between the rings 135a and 135b, and an area 139b between the rings 135b and 135c provide a site for securing the graft (harvested) vessel to the device with lateral sutures, respectively. The stent member 130 also has a thickness that is non-uniform along the axis A of the stent member 130. In the embodiment shown in FIG. 1, the thickness has a minimal thickness of about 0.1 mm. The stent member 130 can be formed with other dimension parameters.

The base member 110 and the stent member 130 are formed such that the lumen of the stent member 130 is in fluid communication with the recess 120 of the base member 110 through the opening 122 in the base body portion 113 of the base member 110.

Moreover, the anastomosis device 100 includes a layer (not shown) of chemical composition coated at least on a part of the first surface 115 of the base member 110 and the inner surface 136a of the stent member 130, respectively, where the coated chemical composition is releasable for reducing restenosis and thrombosis simultaneously. In one embodiment, the layer of chemical composition includes a cytostatic drug component. The cytostatic drug component comprises sirolimus, heparin, tacrolimus or any combination thereof. For example, sirolimus stops cell proliferation without killing cells, thereby minimizing the risk of tissue pathology. The chemical composition is firmly attached to the device via covalent bonding. Other chemical compositions such as other cytostatic drug that may selectively stop cell proliferation by inhibiting cell-cycle progression can also be used to practice the current invention.

All of edges of the anastomosis device 100 are preferred to be rounded so as not to puncture inner surfaces of vessels in which the anastomosis device is placed. Again, other forms of edges can be utilized to practice the present invention.

In operation, the stent member 130 of the anastomosis device 100 is inserted into the graft vessel 150 from a free open end 155 of the graft vessel 150, as shown in FIG. 2A. Then at least one suture is placed in a circular fashion around the graft vessel 150 at an area 139a between the protruded rings 135a and 135b, or an area 139b between the protruded rings 135b and 135c, which prevents the graft vessel 150 from detaching from the stent member 130. The stent member 130 is designed such that when the stent member 130 is inserted into the graft vessel 150, the lumen 133a of the stent member 130 is approximately coincident with a lumen 140 of the graft vessel 150. Once the graft vessel 150 is securely attached to the stent member 130 of the anastomosis device 100, the base member 110 of the anastomosis device 100 is introduced into the target vessel 160, such as a coronary artery, from the opening 165 in the wall of the target vessel 160, as shown in FIG. 2B. The base member 110 is designed to be longer than the dimension of the opening 165 in the wall of the target vessel 160 such that when the base member 110 is introduced into the target vessel 160 from the opening 165 in the wall of the target vessel 160, a force 168 directing outwardly from a central axis 162 of the target vessel 160 is perpendicularly applied to the first surface 115 of the base member 110 to bring the second surface 116 of the base member 110 into contact with an inner surface of the wall of the target vessel 160.

The graft vessel 150 and the target vessel 160 are now stabilized in close contact with one another and sutured together or attached with biologic glue such that in operation, the lumen 140 of the graft vessel 150 is in fluid communication with a lumen 145 of the target vessel 160 through the device 100, as shown in FIG. 2B. Preferably, a biologic glue is used to secure the graft vessel to the target vessel. The biologic glue, such as BioGlue® (Cryolife, Inc., Kennesaw, Ga.), is of high strength, which is stronger than sutures, and can form a non-toxic bond to vascular and cardiac tissues in 30 seconds or less. The BioGlue® can also form strong bonds to leather, rubber, Dacron®, Teflon®, as well as metals. This particular combination of properties is preferably for the CABG environment [6, 7]. Other biologic glues can also be employed to practice the current invention.

When such a device is introduced into a coronary artery (target vessel), it serves as a scaffold for the anastomosis site and functions as a stent to provide support for the coronary artery during and after anastomosis. Due to the introduction of the anastomosis device (a foreign object), intimal hyperplasia and/or thrombogenicity such as a blood clot may be formed in the coronary artery. By slowly releasing a cytostatic drug component, such as sirolimus, heparin, tacrolimus or any combination of them, coated on the device to its surroundings as to administrate cell growth therein, this intimal hyperplasia and/or thrombogenicity can be prevented from happening in the lumens of the donor vessel, such as saphenous vein or internal thoracic artery, and the coronary artery, through endothelial cell seeding. The drug coating on the anastomosis device acts as a local insurance policy. As set forth aboce, a preferable coating drug includes a therapeutic, cytostatic drug component that may selectively stop cell proliferation by inhibiting cell-cycle progression. Various types of therapeutic, cytostatic drug component are available, including sirolimus, heparin, or tacrolimus, to practice the present invention. For example, either sirolimus or heparin freely interacts with the bloodstream and stops cell proliferation without killing cells, thereby minimizing the risk of tissue pathology. Other types of the therapeutic, cytostatic drug components can also be used to practice the current invention. Using of a sirolimus drug coating on stents to inhibit percutaneous target lesion revascularization, thrombosis, and cardiac death has been reported by Regar et al [8].

By combining endothelial cell seeding of the outer surface of the anastomosis device with a coated chemical composition, such as a cytostatic drug component that may selectively stop cell proliferation by inhibiting cell-cycle progression, the device, in operation, not only has a therapeutic, cytostatic drug-eluting function to inhibit restinosis (reblockage) in the lumens of the donor vessel and the coronary artery, but also encourages healing and fusion of the donor vessel and the coronary artery at the site where the donor vessel and the coronary artery meet through endothelial cell seeding. The device is further configured such that the drug-eluting function does not override the healing process.

The anastomosis device can be made of a biocompatible material that ensures the device to withstand the pressures and the flow rates associated with a beating heart and serves as a stent by continuing to provide support and insuring patency in the graft vessel and the target vessel during and after the anastomosis. Based on experimentations, the maximum pressure inherent in the beating heart system that affects the fidelity of the device is 560 mmHg or 7.46×10⁴ N/m². Materials including titanium, stainless steel, tantalum, or the like, meet the requirements and can be used to make the device. One of preferable materials for making the device is Polyetheretherketone™ (PEEK™) polymer, which combines strength, purity, chemical resistance, and ease of processing with superb sterilization resistance, a lack of interaction with biological systems, and a lack of interaction with a magnetic field. The device can be formed in a solid form, a porous form, a meshwork form, or any combination thereof. In one embodiment, the device is formed to be porous enough to allow contact between the blood stream and the walls of the vasculature, which prevents ischemic tissue from developing at the anastomosis site.

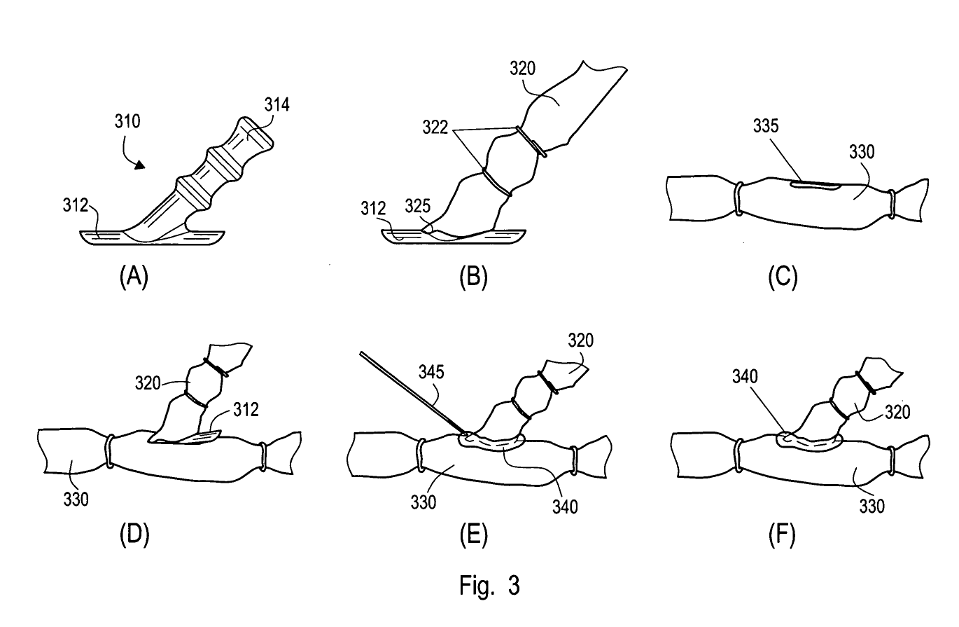

In another aspect, the present invention relates to a method for anastomosing a free end of a graft vessel to a wall of a target vessel, where each of the graft vessel and the target vessel has a lumen formed therethrough. Referring now to FIG. 3, the method includes the following steps: at first, an anastomosis device 310 is provided. As shown in FIG. 3A, the device 310 includes a base member 312 and a stent member 314 projecting away from the base member 312. Secondly, the stent member 314 of the device 310 is inserted into the graft vessel 320 from a free end 325 of the grafted vessel 320, whereby the lumen of the stent member 314 is approximately coincident with the lumen of the graft vessel 320, as schematically shown in FIG. 3B. In one embodiment, the graft vessel 320 is laterally sutured in a circular fashion around the stent member 314 of the device 310 at positions 322. Next, an opening 335 is incised in the wall of the target vessel 330 at an anastomosis site to which the graft vessel 320 is secured, as shown in FIG. 3C. Then the base member 312 of the device 310 is introduced into the target vessel 330 through the opening 335 in the wall of the target vessel 330 so as to bring the free end 325 of the graft vessel 320 in close contact with the wall of the target vessel 330, as shown in FIG. 3D. Finally, the free end 325 of the graft vessel 320 is secured to the wall of the target vessel 330 at the anastomosis site, such that in operation, the lumen of the graft vessel 320 is in fluid communication with the lumen of the target vessel 330 through the device 310. In one embodiment, the free end 325 of the graft vessel 320 is secured to the wall of the target vessel 330 with biocompatible glue 340 with a standard applicator gun and/or tip 345, as shown in FIGS. 3E and 3F, respectively. In another embodiment, the free end 325 of the graft vessel 320 is secured to the wall of the target vessel 330 with suture.

The device can at least be partially coated with a layer of chemical composition which may be slowly released to its surrounding environment, where the coated chemical composition contains a cytostatic drug such as sirolimus, heparin, or tacrolimus. Other chemical compositions such as other cytostatic drug that may selectively stop cell proliferation by inhibiting cell-cycle progression can also be used to practice the current invention.

Without intend to limit the scope of the invention, further exemplary procedures and experiments of the same according to the embodiments of the present invention are given below.

In Vitro Experiment: Anastomoses were made between cryopreserved human saphenous vein segments and coronary arteries in vitro on either excised bovine or porcine hearts using the invented anastomosis device as disclosed in the application.

A number of bovine or porcine hearts were acquired and the coronary arteries were cleaned with a saline-heparin solution. Thawed, cryopreserved segments of human sephanous vein were used as a bypass vessel. The saphenous vein has a first free open end and an opposite, second free open end. In brief, the saphenous vein and a left anterior descending (hereinafter “LAD”) coronary artery were positioned in a close proximity by the anastomosis device of the present invention. First, a stent member of the anastomosis device was inserted into the first free open end of the saphenous vein and secured in place using BioGlue® or suture. Suture was placed in a circular fashion around the saphenous vein between the protruded rings on the stent member, which ensured mechanical stability to prevent the saphenous vein from detaching from the anastomosis device. BioGlue® was applied to a proximal region of the stent member of the anastomosis device where the saphenous vein was attached to the artery surface. Next, an opening of a reasonable size was incised in the coronary artery. A base member of the anastomosis device then was inserted into the coronary artery from the incised opening. The saphenous vein and the coronary artery were now stabilized in close contact with one another and were easily sutured together or attached with BioGlue®. Preferably, BioGlue® was used to secure the saphenous vein to the coronary artery, with its standard applicator gun and tip and allowed to set for about 2 minutes.

Along the first free open end of the saphenous (harvested-bypass) vein, a standard vein graft cannula was fitted and connected to a pressure-transducer box. Saline solution was then injected into the saphenous vein through the first free open end. And a flow of the injected saline solution through the second free open end of the saphenous vein was confirmed. After the flow through the saphenous vein was assured, the second free open end of the saphenous vein was clipped. A large syringe was then used to forcefully inject saline solution at pressures of at least 300 mmHg into the saphenous vein and coronary arteries. The large syringe was connected in a Y fashion to the first free open end of the saphenous vein and the pressure transducer. Flow through the proximal artery was confirmed via saline solution proceeding from the coronary ostia and from the distal artery by slicing the heart in a bread-loaf slice close to the apex and assessing runoff. Upon completion of the in vitro experiments, in vivo anastomoses using the device was conducted to test the viability in a living subject.

In Vivo Experiment: In vivo anastomoses were performed in the off-pump coronary artery bypass grafting using the invented anastomosis device as described above. An exposed coronary artery, typically a LAD coronary artery, was immobilized by the FLEXSITE® tissue stabilizer incorporating a novel turnbuckle vacuum foot mechanism as described above. Coronary artery anastomoses were performed, and evaluated both intra-operatively and post-operatively. Coronary hyperemic responses were determined in relation to a mean baseline blood pressure during and after the anastomosis. The graft vessel flow was also monitored. Methods to confirm a patency of the anastomosis included angiography, flow measurement, and histological analysis of the tissue at the anastomosis. Time of flight angiography permitted tagging of blood in one region of the vasculature surrounding the anastomosis and detection of it in another. Sectioning and staining, in histological analysis, allowed detection of intimal hyplerplasia and determine the degree to which it affects the walls of the anastomosis. Together, all of these tests confirmed the patency of the anastomosis.

In one embodiment, a set of ten mongrel (beagle) female dogs, ex-breeders were used to practice the current invention. A human patient and/or various types of animals, or other numbers of the dogs can also be employed to practice the present invention. These dogs were chosen to have an age averagely about 6.6 years and a weight averagely about 15.6 kg. These dogs fasted for about 24 hours and pre-medicated intramuscularly with Fentanyl (Janssen Pharmaceutica, Beerse, Belgium) about 50 mg/kg, Dehydrobenzperidol about 2.5 mg/kg and Atropine about 0.5 mg prior to an induction of anesthesia. After a dog was placed in the dorsal decubitus position, a peripheral intravenous line was placed in the left upper limb. Cephalosporin (about 15 mg/kg) was injected intramuscularly for antibiotic prophylaxis and maintenance intravenous fluids was given with crystalloid fluid. A three-lead electrocardiogram was used to monitor heart rate and rhythm of the dog undergoing the anastomosis. Anesthesia started via a halothane-oxygen mixture. The dog was intubated and ventilated with a respirator. All ventilation parameters were adjusted to keep the blood gas values within a predetermined range. Anesthesia was induced intravenously with sodium penthotal about 15 mg/kg and pancurorium about 1 mg/kg and maintained by continuous sodium penthotal about 10 μg/kg and pancurorium about 0.5 mg/kg and halothan gas about 1-2% in air/oxygen. An arterial pressure catheter was inserted into the common femoral artery and a central venous line was placed transcutaneously in the jugular vein. The chest of the dog was shaved, prepared, and draped in a sterile fashion. A distal partial stemotomy (the IV-th to VI-th intercostal space) was performed and prolonged towards the linea alba. The LITA artery was dissected from the first intercostal branch until the distal bifurcation into the ramus epigastricus and musculo-phrenicus. Pericardium was opened and suspended. The LAD coronary artery was dissected and occluded proximal and distal of the future arteriotomy by means of elastic snares.

The in vivo anastomosis was performed between the LITA and a coronary arteriotomy of about 5 mm on the beating heart of the dog. The dog heart was left beating, and a localized stabilization device, attached to the FLEXSITE® arm (Ethicon, Inc., Somerville, N.J.), was positioned to provide a view window of the LAD coronary artery. The FLEXSITE® arm was locked rigidly to stabilize the anastomosis site, and the LAD coronary artery was opened in its mid portion. The turnbuckle mechanism on the localized stabilization device was adjusted by a surgeon to provide lateral tension to the anastomosis site. In the same manner as detailed above, the distal end of the LITA was opened. A sent member of the anastomosis device was inserted into the LITA from the distal end of the LITA and secured in place using BioGlue® or suture. Suture was placed in a circular fashion around the vessel between the protruded rings on the stent member, which added mechanical stability to prevent the vessel from detaching from the device. Once the LITA vessel was securely attached, a base member of the anastomosis device was inserted into the coronary artery. When the anastomosis device was introduced into the coronary artery, an outward force, perpendicular to the coronary artery surface, was applied to the anastomosis device so that the base member of the device was in contact with an inner surface of the coronary artery. The LITA and the coronary artery were now stabilized in close contact with one another in the anastomosis site, then BioGlue® was applied to the anastomosis site with its standard applicator gun and tip. In one embodiment, about 5 cc Bioglue® was applied and dried for about 2 minutes. The distal elastic snare was partially lifted allowing slow progressive retrograde filling of the vessels with certain extra vascular oozing. The distal elastic snare was removed and the LITA-graft vessel was opened. Definitive proximal occlusion of the LAD upstream should exclude competitive flow from the native coronary. After hemostatic control, the stemotomy was closed in layers with insertion of a chest drain. After extubation, the dog was kept in the recovery room for about 4 hours. Care must be taken for catheters, which was progressively removed. Chest drainages were removed after about 2 hours from the operation. Cephalosporin was administered intramuscularly once daily. Diuretics and analgesics would be administered as necessary. After about 4 hours from the operation, all medication given, the dog was returned to a controlled animal facility where the general health of the dog would be checked daily.

These dogs were randomly grouped into groups I and II with each group having five dogs. The dogs of group I were sacrificed after 6 weeks from the anastomosis and the dogs of group II were sacrificed after 3 months from the anastomosis. Ischemia was checked upon by ST-segmental changes on the electrocardiographic (ECG) from induction to 2 hours after the anastomosis procedure, considered positive over 2 mm of ST-elevation, creatine kinase MB (CKMB) enzymes and disrhythmia, in particular, ventricular polymorphism. Angiographic control of the native coronary system and the LAD graft for the patency was performed in 6 weeks post minimally invasive direct coronary artery bypass (MIDCAB). Consequently, the dogs of group I were sacrificed while the dogs of group II were kept alive for another 6 weeks. For histology, the anastomotic site was longitudinal transsected and embedded in paraffin. In one embodiment, about 4 mm thick sections were stained with hematoxylin and eosin, Masson's trichrome for collagen, Von Giesson for elastin and phosphotungsten-acid-hematoxylin for fibrin. Ultrathin sections of the anastomosis was prepared for examination with a scanning electron microscope, for example, a Phillips XL-40 (Philips Electron Optics, Eindhoven, The Netherlands). Tissue specimens were dried by critical point method with CO₂ and covered with gold.

For a long term evaluation of the in vivo anastomosis, one candidate animal is a yearling goat weighing approximately about 50 kg. After a small left anterior thoracotomy was performed, either the left or right internal thoracic artery was harvested. Once it was obtained, this suitable length of artery was ligated distally and then flushed with heparin solution. The pericardium was accessed and opened through the same thoracotomy while purse string sutures were placed on the ascending aorta and the right atrial appendage. Applying the same procedures as disclosed above to the yearling goat, an in vivo anastomosis was conducted between a LITA and a LAD coronary artery via an inferior sternotomy on the beating heart of the yearling goat. Afterwards, a blood flow through the LITA was confirmed and then a Ligaclip was applied to the distal end of the LITA graft, resulting in an end-to-side anastomosis. The proper precautions were taken after the anastomosis was successful and the chest of the goat was closed. In one embodiment, an autopsy of at least one yearling goat was performed in about 24 hours after the in vivo anastomosis so as to assess early results of the in vivo anastomosis. Two or more yearling goats were permitted to recover and autopsies were done in about 10 months and about 1 year after the in vivo anastomosis procedure. These goats were fed a normal diet and received humane care in accordance with the “Guide for the Care and Use of Laboratory Animals” (National Institutes of Health publication 86-23, revised 1985).

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

### REFERENCE LIST

[1]. Coronary Heart Disease, 2002 Heart and Stroke Statistical Update, Dallas, Tex. American Heart Association, 2001.
[2]. Facts About Coronary Heart Disease, National Institutes of Health, 27 Jul. 2001.
[3]. The Heart and Coronary Artery Disease, Hall-Garcia Cardiology Associates, July 2001.
[4]. Sundt T. M.,Coronary Artery Bypass Grafting, The Society of Thoracic Surgeons Patient Information Multimedia, 2003.
[5]. Arom K., et al., Safety and efficacy of off-pump coronary artery bypass grafting, Annals of Thoracic Surgery, 2000;69:704-710
[6]. Kowanko N., Adhesive composition and method, U.S. Pat. No. 5,385,606, 1995.
[7]. Gundry S. R., Black K, Izutanii H., Sutureless artery bypass with BioGlue® anastomoses: preliminary in vivo and in vitro results, J Thorac and Cardiovasc Surg, 2000;120:473-477.
[8]. Regar E, Serruys P. W., Bode C, Morice M. C., et al., Angiographic findings of the multicenter Randomized Study With the Sirolimus-Eluting Bx Velocity Balloon-Expandable Stent (RAVEL): sirolimus-eluting stents inhibit restenosis irrespective of the vessel size, Circulation® 2002 American Heart Association, Inc., 2002;106:1949-1956.

1. An anastomosis device for connecting a free open end of a graft vessel to a wall of a target vessel, comprising:
a. a base member having a first surface, a second surface, a semicircular cylindrical wall formed therebetween the first surface and the second surface and an opening formed in the semicircular cylindrical wall;
b. a stent member having an inner surface, an outer surface, a cylindrical wall formed therebetween the inner surface and the outer surface and a lumen formed by the inner surface, and outwardly projecting away from the second surface of the base member along a direction having an angle, α, relative to the base member such that the lumen of the stent member is in fluid communication with the opening in the semicircular cylindrical wall of the base member; and
c. a layer of chemical composition coated at least on a part of the first surface of the base member and the inner surface of the stent member,
wherein, in operation, the stent member is inserted in the graft vessel through the free open end and the base member is introduced into the target vessel from an opening in the wall of the target vessel so as to anastomose the free open end of the graft vessel to the wall of the target vessel such that a lumen of the graft vessel is in fluid communication with a lumen of the target vessel through the device, and the coated chemical composition is gently released to its surrounding environment.

2. The device of claim 1, wherein the base member further has a first end and a second end defining a base length, each of the first end and the second end of the base member rounded with a radius.

3. The device of claim 2, wherein the base member is adapted such that when the base member is introduced into the target vessel, a force directing outwardly from a central axis of the target vessel is perpendicularly applied to the first surface of the base member to bring the second surface of the base member into contact with an inner surface of the wall of the target vessel.

4. The device of claim 1, wherein the stent member further comprises a number of rings protruding outwardly from the outer surface of the stent member with each ring at a predetermined position.

5. The device of claim 4, wherein each of the number of rings has a semicircular cross-section with a diameter.

6. The device of claim 1, wherein the coated chemical composition is adapted for reducing restenosis and thrombosis simultaneously.

7. The device of claim 6, wherein the coated chemical composition comprises a cytostatic drug component.

8. The device of claim 7, wherein the cytostatic drug component comprises one of sirolimus, heparin, tacrolimus, and any combination thereof.

9. The device of claim 1, wherein the device is formed of a biocompatable material.

10. The device of claim 9, wherein the device is formed in one of a solid form, a porous form, a meshwork form, and any combination thereof.

11. The device of claim 1, wherein the free open end of the graft vessel is secured to the wall of the target vessel at an anastomosis site in which the device is placed with biocompatable glue.

12. The device of claim 1, wherein the free open end of the graft vessel is secured to the wall of the target vessel at an anastomosis site in which the device is placed with suture.

13. An anastomosis device, comprising:
a. a base member having a first surface, a second surface, a base wall formed therebetween the first surface and the second surface, and an opening formed in the base wall; and
b. a stent member having an inner surface, an outer surface, a tubular wall formed therebetween the inner surface and the outer surface and a lumen formed by the inner surface, and outwardly projecting away from the second surface of the base member along a direction having an angle, α, relative to the base member such that the lumen of the stent member is in fluid communication with the opening in the base wall of the base member.

14. The device of claim 13, wherein the base wall of the base member is formed in a semicircular cylindrical form.

15. The device of claim 13, wherein the base wall of the base member is formed in a circular cylindrical form.

16. The device of claim 13, wherein the base wall of the base member has a thickness defined therebetween the first surface and the second surface of the base member that is uniform.

17. The device of claim 13, wherein the tubular wall of the stent member has a thickness defined therebetween the inner surface and the outer surface of the stent member that is non-uniform along the projecting direction.

18. The device of claim 13, wherein the device is formed of a biocompatable material.

19. The device of claim 18, wherein the device is formed in one of a solid form, a porous form, a meshwork form and any combination thereof.

20. The device of claim 18, wherein the device is at least partially coated with a layer of chemical composition.

21. The device of claim 20, wherein the coated chemical composition is adapted for reducing restenosis and thrombosis simultaneously.

22. The device of claim 21, wherein the coated chemical composition comprises a cytostatic drug component.

23. The device of claim 22, wherein the cytostatic drug component comprises one of sirolimus, heparin, tacrolimus, and any combination thereof.

24. An anastomosis device, comprising:
a. a base member having a first end, a second end, and a body portion with a length defined between the first end and the second end, wherein the body portion is formed with a first edge portion and a second edge portion, the first edge portion and the second edge portion being apart from each other defining a recess therebetween, and an opening thereof; and
b. a stent member having a substantially tubular body with a longitudinal axis, A, and a lumen defined by the substantially tubular body, and projecting away from the base member such that to form an angle, α, between the base member and the longitudinal axis, A, of the substantially tubular body, wherein the lumen of the stent member is in fluid communication with the recess of the base member through the opening.

25. The device of claim 24, further comprising a layer of chemical composition coated at least on a part of the base member and the stent member, respectively, wherein the coated chemical composition is releasable for reducing restenosis and thrombosis simultaneously.

26. The device of claim 25, wherein the coated chemical composition comprises a cytostatic drug component.

27. The device of claim 26, wherein the cytostatic drug component comprises one of sirolimus, heparin, tacrolimus, and any combination thereof.

28. The device of claim 24, wherein the angle, α, is greater than zero but smaller than 180°.

29. The device of claim 24, wherein the stent member further comprises one or more rings protruding outwardly from the outer surface of the stent member.

30. The device of claim 24, wherein the body portion of the base member with a curvature has a semicircular cross-section.

31. An anastomosis device, comprising:
a. means for performing an anastomosis to connect a wall of a target vessel in fluid communication with a lumen in the target vessel through an opening in the wall of the target vessel; and
b. means for releasing a chemical composition for reducing restenosis and/or thrombosis during and/or after the anastomosis.

32. The device of claim 30, wherein the means for performing an anastomosis comprises
a. a base member having a first end, a second end, and a body portion with a length defined between the first end and the second end, wherein the body portion is formed with a first edge portion and a second edge portion, the first edge portion and the second edge portion being apart from each other defining a recess therebetween, and an opening thereof; and
b. a stent member having a substantially tubular body with a longitudinal axis, A, and a lumen defined by the substantially tubular body, and projecting away from the base member such that to form an angle, α, between the base member and the longitudinal axis, A, of the substantially tubular body, wherein the lumen of the stent member is in fluid communication with the recess of the base member through the opening.

33. The device of claim 31, wherein the means for releasing a chemical composition comprises a layer of chemical composition coated at least on a part of the base member and the stent member, respectively, wherein the coated chemical composition is releasable for reducing restenosis and thrombosis simultaneously.

34. The device of claim 33, wherein the coated chemical composition comprises a cytostatic drug component.

35. The device of claim 34, wherein the cytostatic drug component comprises one of sirolimus, heparin, tacrolimus, and any combination thereof.

36. The device of claim 32, wherein the angle, α, is greater than zero but smaller than 180°.

37. The device of claim 32, wherein the stent member further comprises one or more rings protruding outwardly from the outer surface of the stent member.

38. The device of claim 32, wherein the body portion with a curvature has a semicircular cross-section.

39. A method for anastomosing a free end of a graft vessel to a wall of a target vessel, wherein the graft vessel has a lumen formed therethrough and the target vessel has a lumen formed therethrough, comprising the steps of:
a. providing a device having a base member having an opening and a stent member having a lumen formed therethrough and projecting away from the base member such that the lumen of the stent member is in fluid communication with the opening in the base member;
b. inserting the stent member of the device into the graft vessel through the free end of the grafted vessel, whereby the lumen of the stent member is approximately coincident with the lumen of the graft vessel;
c. incising an opening in the wall of the target vessel at an anastomosis site to which the graft vessel is secured;
d. introducing the base member of the device into the target vessel through the opening in the wall of the target vessel such that the lumen of the target vessel is in fluid communication with the lumen of the stent member of the device through the opening in the base wall of the base member of the device; and
e. securing the free end of the graft vessel to the wall of the target vessel at the anastomosis site,
wherein, in operation, the lumen of the graft vessel is in fluid communication with the lumen of the target vessel through the device.

40. The method of claim 39, wherein the device is at least partially coated with a layer of chemical composition.

41. The method of claim 40, further comprising the step of slowly releasing the coated chemical composition from the device to its surrounding environment.

42. The method of claim 41, wherein the coated chemical composition is adapted for reducing restenosis and thrombosis simultaneously.

43. The method of claim 42, wherein the coated chemical composition comprises a cytostatic drug component.

44. The method of claim 43, wherein the cytostatic drug component comprises one of sirolimus, heparin, tacrolimus, and any combination thereof.

45. The method of claim 39, wherein the inserting step comprises the step of laterally suturing the graft vessel in a circular fashion around the stent member of the device.

46. The method of claim 39, wherein the securing step is performed with biocompatible glue.

47. The method of claim 39, wherein the securing step is performed with suture.
